(19) United States
(12) Patent Application Publication
(10) Pub. No.: US 2023/0357308 A1
(43) Pub. Date: Nov. 9, 2023

(54) CRYSTALLINE SODIUM CHOLESTERYL SULFONATE

(71) Applicant: Durect Corporation, Cupertino, CA (US)

(72) Inventors: WEIQI LIN, Emerald Hills, CA (US); MICHAEL HOLTZ-MULHOLLAND, Lachine, Quebec (CA); JULIETTE SABBATANI, Montreal, Quebec (CA); JING TENG, West Lafayette, IN (US)

(21) Appl. No.: 18/028,446

(22) PCT Filed: Oct. 11, 2021
(86) PCT No.: PCT/US2021/054420

§ 371 Date: Mar. 24, 2023

Related U.S. Application Data
(60) Provisional application No. 63/090,589, filed on Oct. 12, 2020.

(51) Int. Cl. C07J 31/00

(52) U.S. Cl. CPC C07J 31/00; C07B 2200/13

(57) ABSTRACT

Crystalline and non-crystalline forms of crystalline sodium cholesteryl sulfonate are disclosed herein. Such forms include crystalline sodium sulfonate Form F and crystalline sodium cholesteryl sulfonate Form J as well as non-crystalline sodium cholesteryl sulfonate Form A. Processes for making such crystalline and non-crystalline forms and methods of treating disease with such forms are further provided.

### CROSS REFERENCE TO RELATED APPLICATIONS

This application claims priority to U.S. Provisional Application No. 63/090,589, filed Oct. 12, 2020, which is incorporated herein by reference.

### BACKGROUND

It has been shown previously that nuclear cholesterol metabolite Sodium; (3S,8S,9S,10R,13R,14S,17R)-17-[(1R)-1,5-dimethylhexyl]-10,13-dimethyl-2,3,4,7,8,9,11,12,14,15,16,17-dodecahydro-1H-cyclopenta[a]phenanthrene-3-sulfonate (“sodium cholesteryl sulfonate”) decreases lipid biosynthesis and increases cholesterol secretion and degradation, and may be useful for the treatment and prevention of hypercholesterolemia, hypertriglyceridemia, and conditions related to fat-accumulation and inflammation (e.g. non-alcoholic fatty liver disease (NAFLD), non-alcoholic steatohepatitis (NASH), alcoholic hepatitis, acute kidney injury (AKI), acute lung injury (ALI), multi-organ injury, metabolic disorders/disease, diabetes, psoriasis, and atherosclerosis).

Cholesterol is used by the body for the manufacture and repair of cell membranes, and the synthesis of steroid hormones and vitamin D, and is transformed to bile acids in the liver. There are both exogenous and endogenous sources of cholesterol. The average American consumes about 450 mg of cholesterol each day and produces an additional 500 mg to 1,000 mg in the liver and other tissues. Another source is the 500 mg to 1,000 mg of biliary cholesterol that is secreted into the intestine daily, and about 50 percent is reabsorbed (enterohepatic circulation).

High serum lipid levels (hypercholesterolemia and hypertriglyceridemia) are associated with the accumulation of cholesterol in arterial walls and can result in NAFLD and atherosclerosis. The plaques that characterize atherosclerosis inhibit blood flow and promote clot formation and can ultimately cause death or severe disability via heart attacks and/or stroke. A number of therapeutic agents for the treatment of hyperlipidemia have been developed and are widely prescribed by physicians. Unfortunately, only about 35% of patients are responsive to the currently available therapies.

Non-alcoholic fatty liver disease (NAFLD) is the most common liver disease in the United States. This condition is associated with obesity, type-II adult onset diabetes, sedentary lifestyle, and diets high in fat. The earlier stage of NAFLD, fatty liver, is potentially reversible when proper treatment steps are taken. However, left unchecked, it can progress to inflammation of liver cells (non-alcoholic steatohepatitis, or NASH) which is much more difficult to treat. Without treatment, NASH can result in irreversible scarring of liver tissue (steatonecrosis), with the potential to cause cirrhosis, liver failure, and liver cancer.

Crystalline solids are generally more favorable for processing, storage, and stability than non-crystalline amorphous solids, for example. However, energetics may not favor the ready formation of suitable crystalline solids and polymorphism may make creating stable crystalline solids of a particular active pharmaceutical ingredient impractical. Herein, the inventors disclose crystalline sodium cholesteryl sulfonate.

### SUMMARY

In some aspects of the present disclosure, crystalline sodium cholesteryl sulfonate is provided.

In further aspects of the disclosure, non-crystalline sodium cholesteryl sulfonate is provided.

In still further aspects of the disclosure, mesophases of sodium cholesteryl sulfonate are provided.

In other aspects of the present disclosure, stable non-crystalline sodium cholesteryl sulfonate is provided.

In other aspects of the present disclosure, stable crystalline sodium cholesteryl sulfonate is provided.

In other aspects of the present disclosure, hydrates of crystalline sodium cholesteryl sulfonate are provided.

In other aspects of the present disclosure, hydrates of non-crystalline sodium cholesteryl sulfonate are provided.

In yet additional aspects of the disclosure, solvates of crystalline sodium cholesteryl sulfonate are provided.

In yet additional aspects of the disclosure, solvates of non-crystalline sodium cholesteryl sulfonate are provided.

In additional aspects of the present disclosure, sodium cholesteryl sulfonate Form A, crystalline sodium cholesteryl sulfonate Form F, and crystalline sodium cholesteryl sulfonate Form J are provided.

In yet additional aspects of the present disclosure, mixtures of two or more solid forms of sodium cholesteryl sulfonate including crystalline sodium cholesteryl sulfonate are provided.

In other aspects of the present disclosure, methods of treating or preventing one or more of hypercholesterolemia, hypertriglyceridemia, and conditions related to fat-accumulation and inflammation, for example, non-alcoholic fatty liver disease (NAFLD), non-alcoholic steatohepatitis (NASH), alcoholic hepatitis, acute kidney injury (AM), acute lung injury (ALI), multi-organ injury, metabolic disorders/disease, diabetes, psoriasis, or atherosclerosis, comprising administering to a patient in need thereof an effective amount of one or more of (i) sodium cholesteryl sulfonate Form A, crystalline sodium cholesteryl sulfonate Form F, and crystalline sodium cholesteryl sulfonate Form J or (ii) pharmaceutical compositions comprising one or more of sodium cholesteryl sulfonate Form F, and crystalline sodium cholesteryl sulfonate Form J are provided.

In further aspects of the present disclosure, pharmaceutical compositions comprising one or more of sodium cholesteryl sulfonate Form A, crystalline sodium cholesteryl sulfonate Form F, and crystalline sodium cholesteryl sulfonate Form J are provided.

In other aspects of the present disclosure, processes for making one or more of sodium cholesteryl sulfonate Form A, crystalline sodium cholesteryl sulfonate Form F, and crystalline sodium cholesteryl sulfonate Form J are provided.

In further aspects of the disclosure, use of one or more of sodium cholesteryl sulfonate such as crystalline sodium cholesteryl sulfonate Form A, crystalline sodium cholesteryl sulfonate Form F, or crystalline sodium cholesteryl sulfonate Form J and optionally one or more pharmaceutically acceptable excipients, for treating a host mammal with hypercholesterolemia, hypertriglyceridemia, and conditions related to fat-accumulation and inflammation, for example, non-alcoholic fatty liver disease (NAFLD), non-alcoholic steatohepatitis (NASH), alcoholic hepatitis, acute kidney injury (AM), acute lung injury (ALI), multi-organ injury, metabolic disorders/disease, diabetes, psoriasis, or atherosclerosis are provided.

### BRIEF DESCRIPTION OF THE FIGURES

FIG. 1 is a peak-picked x-ray powder diffraction pattern of sodium cholesteryl sulfonate Form A of the full diffractogram.

FIG. 2 is a peak-picked x-ray powder diffraction pattern of crystalline sodium cholesteryl sulfonate Form F.

FIG. 3 is the indexing solution of crystalline sodium cholesteryl sulfonate Form F.

FIG. 4 is a DSC thermogram of crystalline sodium cholesteryl sulfonate Form F.

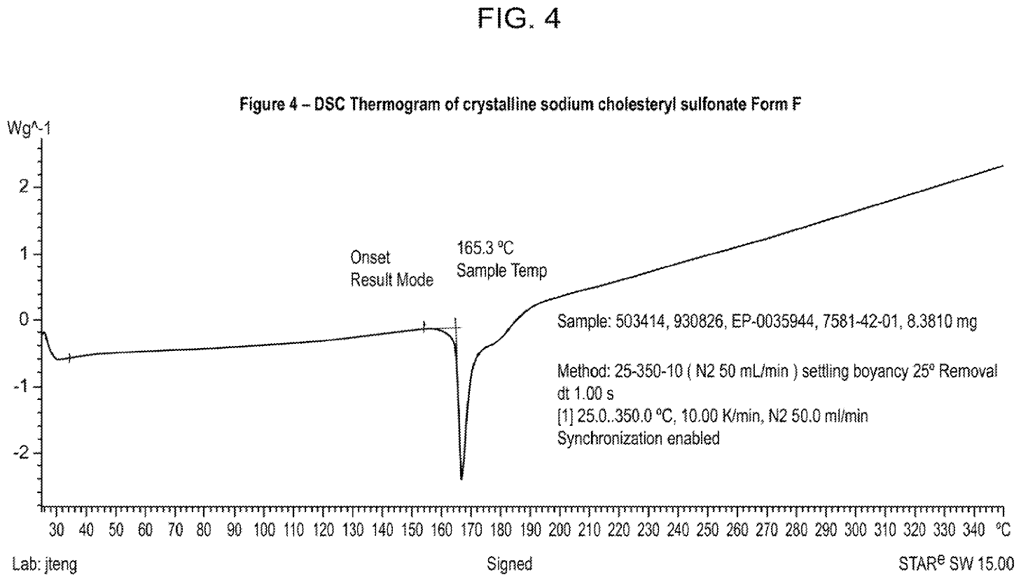

FIG. 5 is a peak-picked x-ray powder diffraction pattern of crystalline sodium cholesteryl sulfonate Form J.

FIG. 6 is a DSC thermogram of sodium cholesteryl sulfonate Form A.

### DETAILED DESCRIPTION

Crystalline sodium cholesteryl sulfonate is readily analyzed by x-ray powder diffraction. An x-ray powder diffraction pattern is an x-y graph with °2θ (diffraction angle) on the x-axis and intensity on the y-axis. The pattern contains peaks which may be used to characterize crystalline sodium cholesteryl sulfonate. The peaks are usually represented and referred to by their position on the x-axis. Unless otherwise specified, peaks are referred to by their position on the x-axis and not their y-axis intensity.

The data from x-ray powder diffraction may be used in multiple ways to characterize crystalline forms. For example, the entire x-ray powder diffraction pattern output from a diffractometer may be used to characterize crystalline sodium cholesteryl sulfonate. A smaller subset of such data, however, may also be, and typically is, suitable for characterizing crystalline sodium cholesteryl sulfonate. For example, a collection of one or more peaks from such a pattern may be used to characterize crystalline sodium cholesteryl sulfonate. In the present application, all reported peak values are in °2θ with Cu-Kα radiation, as set forth in Example 4. Indeed, often even a single x-ray powder diffraction peak may be used to characterize such a crystalline form. When crystalline sodium cholesteryl sulfonate herein is characterized by “one or more peaks” of an x-ray powder diffraction pattern and such peaks are listed, what is generally meant is that any combination of the peaks listed may be used to characterize crystalline sodium cholesteryl sulfonate. Further, the fact that other peaks are present in the x-ray powder diffraction pattern, generally does not negate or otherwise limit that characterization.

In some embodiments, data common to more than one solid form may be used to characterize a collection of such forms. For example, the peak at about 8.3°2θ is common in crystalline sodium cholesteryl sulfonate Form J and crystalline sodium cholesteryl sulfonate Form F. Thus, a peak at about 8.3°2θ may be used to characterize at least some solid forms of crystalline sodium cholesteryl sulfonate.

In addition to variability in peak intensity, there may also be variability in the position of peaks on the x-axis. This variability can, however, typically be accounted for when reporting the positions of peaks for purposes of characterization. Such variability in the position of peaks along the x-axis may derive from several sources (e.g., sample preparation, particle size, moisture content, solvent content, instrument parameters, data analysis software, and sample orientation). For example, samples of the same crystalline material prepared under different conditions may yield slightly different diffractograms, and different x-ray instruments may operate using different parameters. These factors may lead to slightly different diffraction patterns from the same crystalline solid.

Due to such sources of variability, it is common to recite x-ray diffraction peaks using the word “about” prior to the peak value in °2θ. For purposes of data reported herein, that value is generally ±0.2°2θ. This generally means that on a well-maintained instrument one would expect the variability in peak measurement to be ±0.2°2θ or less. X-ray powder diffraction peaks cited herein are generally reported with this variability of ±0.2°2θ unless stated otherwise and are generally intended to be reported with such a variability whenever disclosed herein whether the word “about” is present or not, unless context dictates otherwise. Depending on instrument type and calibration, for example, it is possible for an instrument's variability to less than ±0.2°2θ. Accordingly, in certain embodiments, the variability in a peak value or grouping of peak values ±0.1°2θ, or even ±0.05°2θ, rather than ±0.2°2θ.

Thermal methods are another typical technique to characterize solid forms such as salts. Different polymorphs of the same compound often have different endothermic events such as when measured by Differential Scanning Calorimetry. Such events may include melting. As with any analytical technique, melting point determinations are also subject to variability. Common sources of variability, in addition to instrumental variability, are due to colligative properties such as the presence of other solid forms or other impurities within a sample whose melting point is being measured. Common variability for thermal measurements is on the order of ±1° C. and are generally intended to be reported with such a variability whenever disclosed herein whether the word “about” is present or not.

Sodium cholesteryl sulfonate has the following chemical structure:

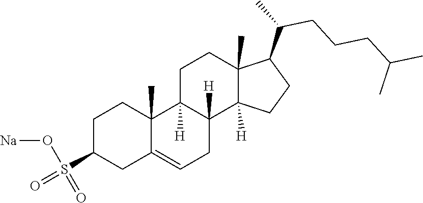

The present disclosure uses the term “Form” to identify different forms of crystalline or non-crystalline of crystalline sodium cholesteryl sulfonate. The differences in the forms can be seen by structure, such as x-ray powder diffraction; properties, such as hygroscopicity or thermal behaviors; and/or both. The use of the term “Form A” means sodium cholesteryl sulfonate Form A. Likewise, Form F means crystalline sodium cholesteryl sulfonate Form F, and Form J means crystalline sodium cholesteryl sulfonate Form J.

Multiple forms, including stable crystalline or non-crystalline forms, of sodium cholesteryl sulfonate are herein reported. These are sodium cholesteryl sulfonate Form A, crystalline sodium cholesteryl sulfonate Form F, and crystalline sodium cholesteryl sulfonate Form J. In the present disclosure, “stable” means that the form does not readily interconvert to another form under a given set of conditions. A metastable form, can, however, so readily convert when exposed to certain conditions. Thus, a form that is stable under one set of conditions (e.g., humidity) may not be stable under another set of conditions. In many embodiments, crystalline and/or non-crystalline sodium cholesteryl sulfonate is provided, including stable crystalline and/or non-crystalline sodium cholesteryl sulfonate.

The term “thermodynamically stable” when comparing forms, means that from a thermodynamic perspective, one form is at a lower energy (the more thermodynamically stable form) than another form. This does not necessarily relate to the rate of conversion between forms since the energy of activation between forms may be sufficiently high that a thermodynamically metastable form is sufficiently stable, however, to be of value. For example, diamond is a thermodynamically metastable form of carbon, compared to graphite, but is sufficiently stable to be of commercial value.

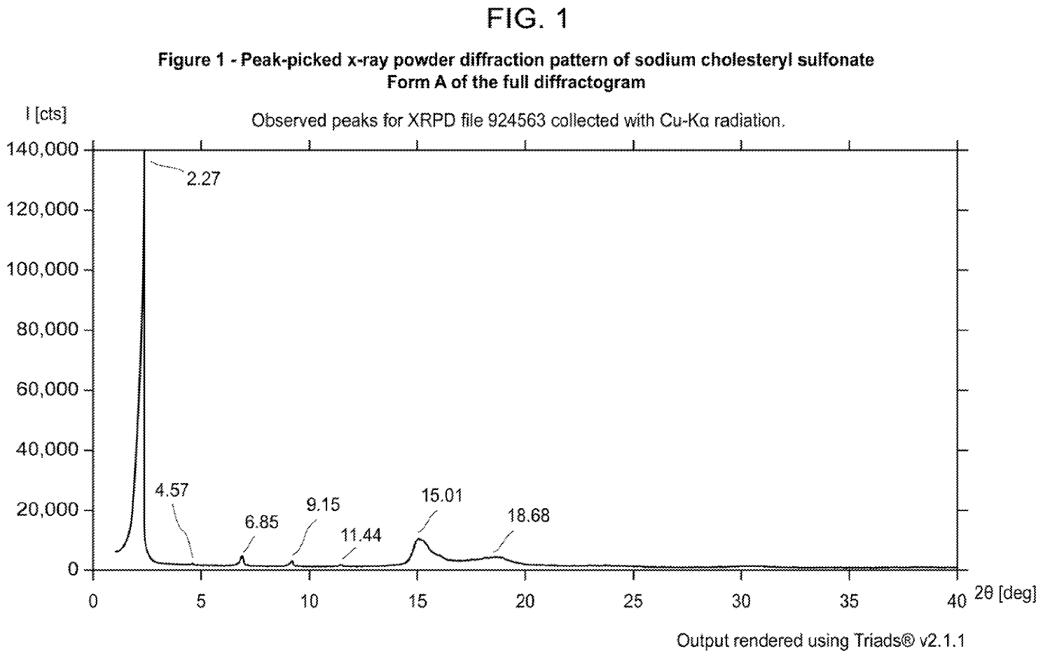

In many embodiments, sodium cholesteryl sulfonate Form A is provided. Without being bound by theory, it is believed that sodium cholesteryl sulfonate Form A is a mesophase. A mesophase is sometimes referred to as a liquid crystal and is often viewed as an intermediate state between crystalline and amorphous forms. A preparation of sodium cholesteryl sulfonate Form A is set forth in Example 1. An x-ray powder diffraction pattern for sodium cholesteryl sulfonate Form A is set forth in FIG. 1 which also indicates specifically identified peaks.

TABLE 1
Observed peaks for Sodium
Cholesteryl Sulfonate Form A

| º2θ | d space (Å) | Intensity (%) |
|---|---|---|
| 2.27 ± 0.20 | 38.817 ± 3.413 | 100 |
| 4.57 ± 0.20 | 19.333 ± 0.846 | 2 |
| 6.85 ± 0.20 | 12.888 ± 0.376 | 3 |
| 9.15 ± 0.20 | 9.656 ± 0.211 | 2 |
| 11.44 ± 0.20 | 7.728 ± 0.135 | 1 |
| 15.01 ± 0.20 | 5.896 ± 0.078 | 8 |
| 18.68 ± 0.20 | 4.745 ± 0.050 | 3 |

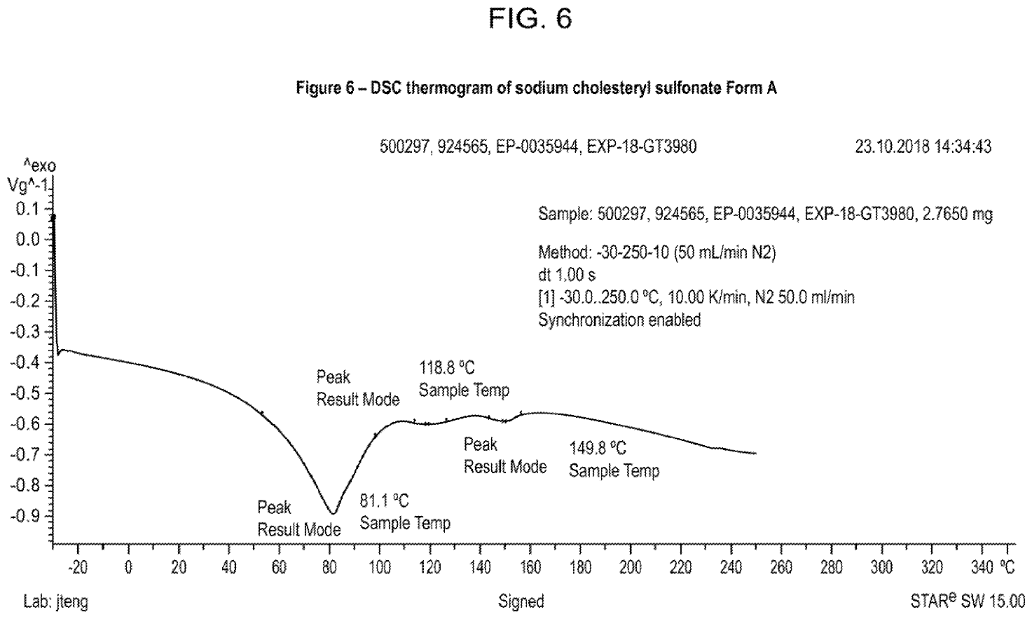

Sodium cholesteryl sulfonate Form A may be characterized by various analytical techniques such as x-ray powder diffraction. The x-ray powder diffraction pattern of sodium cholesteryl sulfonate Form A or portions thereof may be used to characterize sodium cholesteryl sulfonate Form A. For example, sodium cholesteryl sulfonate Form A may be characterized by an x-ray powder diffraction pattern comprising a peak at about 2.3°2θ. Sodium cholesteryl sulfonate Form A may be characterized by a peak in a DSC thermogram at about 81° C. alone or together with an x-ray powder diffraction pattern peak at about 2.3°2θ. In these and other embodiments, sodium cholesteryl sulfonate Form A may be characterized by an x-ray powder diffraction pattern substantially the same as that found in FIG. 1, optionally with the DSC thermogram found in FIG. 6.

Substantially pure sodium cholesteryl sulfonate Form A is further disclosed. “Substantially pure,” as described herein, generally refers to a form herein that is present without any appreciable amounts, other than potentially trace levels of other forms of sodium cholesteryl sulfonate such as crystalline sodium cholesteryl sulfonate. Examples of trace levels include not more than about 10%, 5%, 2%, 1.5%, 1%, 0.5%, 0.25%, 0.1%, or less by weight in total relative to the total amount of sodium cholesteryl sulfonate present.

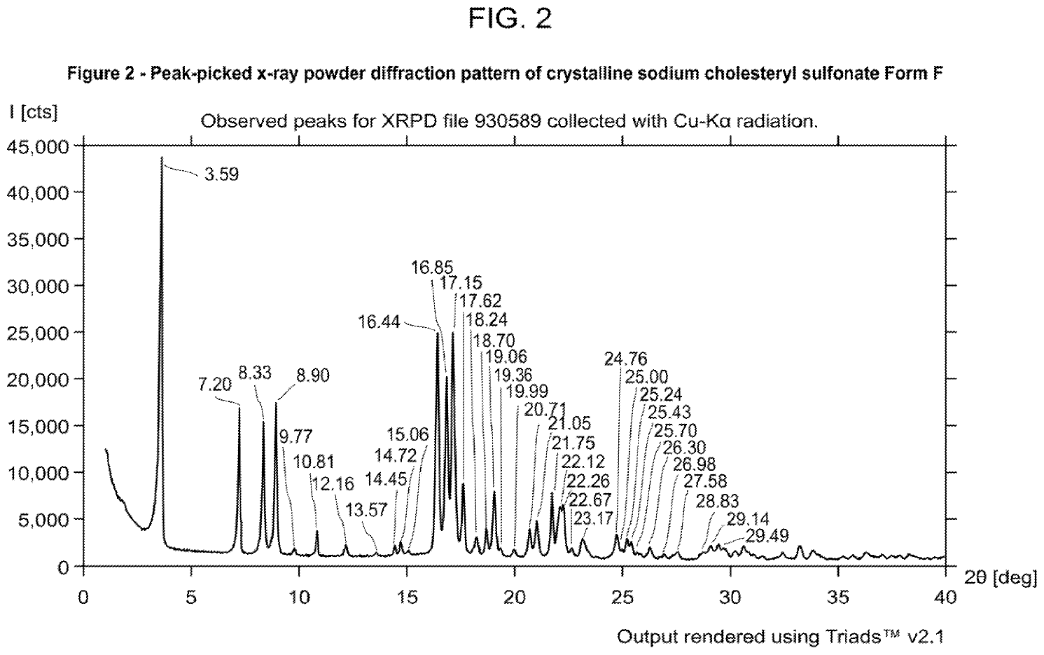

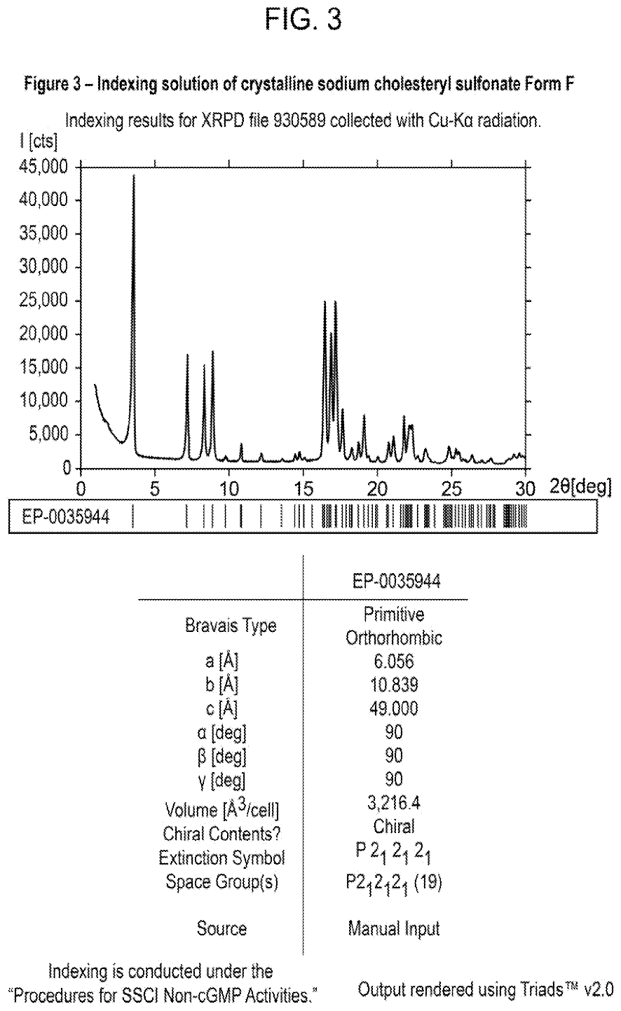

In many embodiments, crystalline sodium cholesteryl sulfonate Form F is provided. Without being bound by theory, it is believed that crystalline sodium cholesteryl sulfonate Form F represents a family of isostructural solvates. For example, it can be prepared from numerous solvents such as dimethylacetamide (“DMA”), dimethylformamide (“DMF”)/H₂O, N-Methyl-2-Pyrrolidone (“NMP”) and NMP/Methyl tert-butyl ether (“MTBE”). An example of a preparation of crystalline sodium cholesteryl sulfonate Form F (FIG. 3), using NMP and MTBE can be found in Example 2. The tentative indexing solution for crystalline sodium cholesteryl sulfonate Form F indicates free volume in the unit cell sufficient for solvent accommodation and ¹H-NMR spectroscopy on crystalline sodium cholesteryl sulfonate Form F prepared using NMP shows 1 mole of NMP per mole of crystalline sodium cholesteryl sulfonate Form F. An x-ray powder diffraction pattern for crystalline sodium cholesteryl sulfonate Form F is set forth in FIG. 2. Table 2 sets forth specifically identified peaks from FIG. 2.

TABLE 2
Observed peaks for Crystalline Sodium
Cholesteryl Sulfonate Form F

| 2θ (º) | d-spacing (Å) | Intensity (%) |
|---|---|---|
| 3.59 ± 0.20 | 24.615 ± 1.372 | 100 |
| 7.20 ± 0.20 | 12.263 ± 0.340 | 39 |
| 8.33 ± 0.20 | 10.603 ± 0.254 | 35 |
| 8.90 ± 0.20 | 9.925 ± 0.223 | 40 |
| 9.77 ± 0.20 | 9.048 ± 0.185 | 4 |
| 10.81 ± 0.20 | 8.175 ± 0.151 | 8 |
| 12.16 ± 0.20 | 7.274 ± 0.119 | 5 |
| 13.57 ± 0.20 | 6.520 ± 0.096 | 3 |
| 14.45 ± 0.20 | 6.127 ± 0.084 | 5 |
| 14.72 ± 0.20 | 6.014 ± 0.081 | 6 |
| 15.06 ± 0.20 | 5.877 ± 0.078 | 4 |
| 16.44 ± 0.20 | 5.388 ± 0.065 | 57 |
| 16.85 ± 0.20 | 5.257 ± 0.062 | 47 |
| 17.15 ± 0.20 | 5.165 ± 0.060 | 58 |
| 17.62 ± 0.20 | 5.030 ± 0.057 | 20 |
| 18.24 ± 0.20 | 4.860 ± 0.053 | 7 |
| 18.70 ± 0.20 | 4.742 ± 0.050 | 9 |
| 19.06 ± 0.20 | 4.652 ± 0.048 | 18 |
| 19.36 ± 0.20 | 4.580 ± 0.047 | 4 |
| 19.99 ± 0.20 | 4.437 ± 0.044 | 4 |
| 20.71 ± 0.20 | 4.286 ± 0.041 | 9 |
| 21.05 ± 0.20 | 4.218 ± 0.040 | 11 |
| 21.75 ± 0.20 | 4.083 ± 0.037 | 18 |
| 22.12 ± 0.20 | 4.016 ± 0.036 | 14 |
| 22.26 ± 0.20 | 3.990 ± 0.035 | 15 |
| 22.67 ± 0.20 | 3.919 ± 0.034 | 4 |
| 23.17 ± 0.20 | 3.835 ± 0.033 | 7 |
| 24.76 ± 0.20 | 3.593 ± 0.029 | 7 |
| 25.00 ± 0.20 | 3.559 ± 0.028 | 4 |
| 25.24 ± 0.20 | 3.526 ± 0.027 | 7 |
| 25.43 ± 0.20 | 3.499 ± 0.027 | 6 |
| 25.70 ± 0.20 | 3.463 ± 0.026 | 3 |
| 26.30 ± 0.20 | 3.385 ± 0.025 | 4 |
| 26.98 ± 0.20 | 3.302 ± 0.024 | 3 |
| 27.58 ± 0.20 | 3.232 ± 0.023 | 3 |
| 28.83 ± 0.20 | 3.095 ± 0.021 | 3 |
| 29.14 ± 0.20 | 3.062 ± 0.021 | 5 |
| 29.49 ± 0.20 | 3.026 ± 0.020 | 5 |

Crystalline sodium cholesteryl sulfonate Form F may be characterized by various analytical techniques such as x-ray powder diffraction. The x-ray powder diffraction pattern of crystalline sodium cholesteryl sulfonate Form F or portions thereof may be used to characterize crystalline sodium cholesteryl sulfonate Form F. For example, crystalline sodium cholesteryl sulfonate Form F may be characterized by an x-ray powder diffraction pattern comprising a peak at about 3.6°2θ. The x-ray powder diffraction pattern may further comprise of one or more peaks at about 7.2°2θ, at about 8.3°2θ, at about 8.9°2θ, at about 9.8°2θ, at about 10.8°2θ, and at about 12.2°2θ.

In these and other embodiments, crystalline sodium cholesteryl sulfonate Form F may be characterized by (i) an x-ray powder diffraction pattern comprising peaks at about 3.6°2θ, at about 7.2°2θ, and at about 8.3°2θ; or (ii) an x-ray powder diffraction pattern comprising peaks at about 3.6°2θ, at about 7.2°2θ, at about 8.3°2θ, and at about 10.8°2θ; or (iii) an x-ray powder diffraction pattern comprising a peak at about 10.8°2θ and one or more peaks at about 3.6°2θ, at about 7.2°2θ, at about 8.3°2θ, at about 8.9°2θ, at about 9.8°2θ, and at about 12.2°2θ; or (iv) an x-ray powder diffraction pattern comprising peaks at about 10.8°2θ, at about 7.2°2θ, and at about 8.3°2θ; or (v) an x-ray powder diffraction pattern comprising peaks at about 10.8°2θ, at about 7.2°2θ, at about 8.3°2θ, and at about 8.9°2θ.

In these and other embodiments crystalline sodium cholesteryl sulfonate Form F may be characterized by a DSC thermogram having an onset temperature of about 165° C. Crystalline sodium cholesteryl sulfonate Form F may further be characterized by an x-ray powder diffraction pattern substantially the same as that found in FIG. 2, optionally with a DSC thermogram onset temperature of about 165° C. There is also a peak which appears in some preparations of crystalline sodium cholesteryl sulfonate Form F at about 2.3°2θ. It is not a part of crystalline sodium cholesteryl sulfonate Form F. In other preparations, a peak at about 15°2θ appears, also not part of crystalline sodium cholesteryl sulfonate Form F.

Substantially pure crystalline sodium cholesteryl sulfonate Form F is further disclosed. “Substantially pure,” as described herein, generally refers to a form herein that is present without any appreciable amounts, other than potentially trace levels of other forms of crystalline sodium cholesteryl sulfonate. Examples of trace levels include not more than about 10%, 5%, 2%, 1.5%, 1%, 0.5%, 0.25%, 0.1%, or less by weight in total relative to the total amount of crystalline sodium cholesteryl sulfonate present.

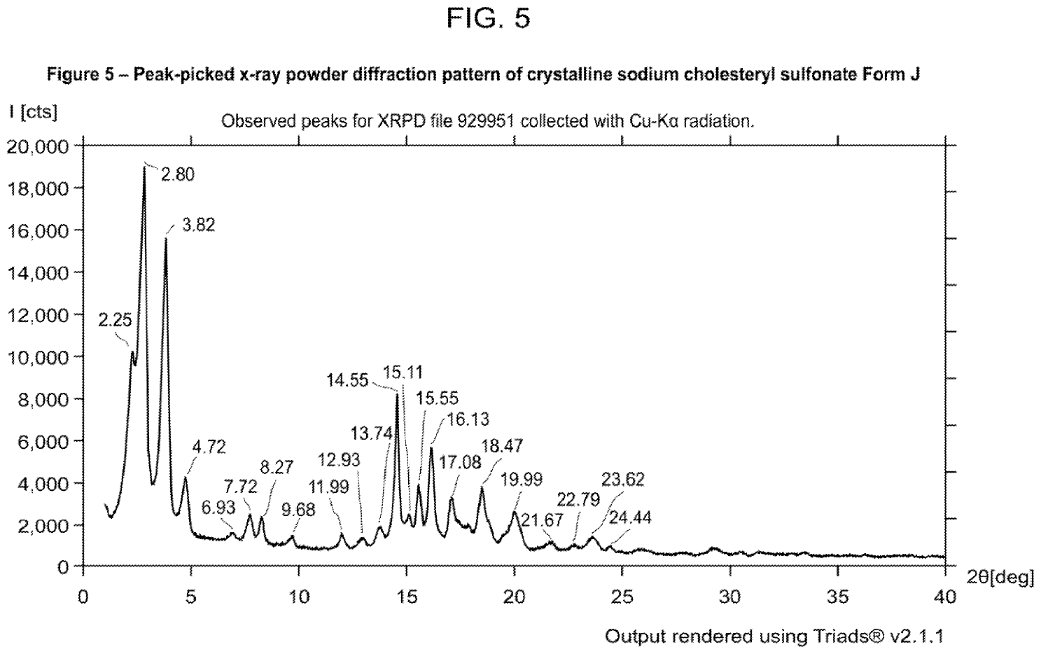

In many embodiments, crystalline sodium cholesteryl sulfonate Form J is provided. Example 3 sets forth a preparation for crystalline sodium cholesteryl sulfonate Form J. An x-ray powder diffraction pattern for crystalline sodium cholesteryl sulfonate Form J is set forth in FIG. 5. Table 3 sets forth specifically identified peaks from FIG. 5.

TABLE 3
Observed peaks for Crystalline
Sodium Cholesteryl Sulfonate Form J

| º2θ | d space (Å) | Intensity (%) |
|---|---|---|
| 2.25 ± 0.20 | 39.154 ± 3.473 | 53 |
| 2.80 ± 0.20 | 31.565 ± 2.257 | 100 |
| 3.82 ± 0.20 | 23.105 ± 1.209 | 81 |
| 4.72 ± 0.20 | 18.718 ± 0.793 | 22 |
| 6.93 ± 0.20 | 12.746 ± 0.367 | 8 |
| 7.72 ± 0.20 | 11.449 ± 0.296 | 13 |
| 8.27 ± 0.20 | 10.686 ± 0.258 | 12 |
| 9.68 ± 0.20 | 9.133 ± 0.188 | 7 |
| 11.99 ± 0.20 | 7.376 ± 0.123 | 8 |
| 12.93 ± 0.20 | 6.840 ± 0.105 | 7 |
| 13.74 ± 0.20 | 6.440 ± 0.093 | 9 |
| 14.55 ± 0.20 | 6.082 ± 0.083 | 42 |
| 15.11 ± 0.20 | 5.861 ± 0.077 | 13 |
| 15.55 ± 0.20 | 5.693 ± 0.073 | 20 |
| 16.13 ± 0.20 | 5.492 ± 0.068 | 29 |
| 17.08 ± 0.20 | 5.188 ± 0.060 | 17 |
| 18.47 ± 0.20 | 4.800 ± 0.052 | 20 |
| 19.99 ± 0.20 | 4.437 ± 0.044 | 13 |
| 21.67 ± 0.20 | 4.097 ± 0.037 | 6 |
| 22.79 ± 0.20 | 3.898 ± 0.034 | 6 |
| 23.62 ± 0.20 | 3.764 ± 0.031 | 7 |
| 24.44 ± 0.20 | 3.640 ± 0.029 | 5 |

Crystalline sodium cholesteryl sulfonate Form J may be characterized by various analytical techniques such as x-ray powder diffraction. The x-ray powder diffraction pattern of crystalline sodium cholesteryl sulfonate Form J or portions thereof may be used to characterize crystalline sodium cholesteryl sulfonate Form J. For example, crystalline sodium cholesteryl sulfonate Form J may be characterized by an x-ray powder diffraction pattern comprising a peak at about 2.8°2θ. The x-ray powder diffraction pattern may further comprise one or more peaks at about 2.3°2θ, at about 3.8°2θ, at about 4.7°2θ, at about 14.6°2θ, and at about 16.1°2θ.

In these and other embodiments, crystalline sodium cholesteryl sulfonate Form J may be characterized by (i) an x-ray powder diffraction pattern comprising peaks at about 2.8°2θ, at about 2.3°2θ, and at about 4.7°2θ; or (ii) an x-ray powder diffraction pattern comprising peaks at about 2.8°2θ, at about 2.3°2θ, at about 4.7°2θ, and at about 2.7°2θ; (iii) or an x-ray powder diffraction pattern comprising peaks at about 2.8°2θ, at about 2.3°2θ, at about 4.7°2θ, at about 2.7°2θ, and at about 14.6°2θ.

In other embodiments, crystalline sodium cholesteryl sulfonate Form J may be characterized by an x-ray powder diffraction pattern substantially the same as that found in FIG. 5.

Substantially pure crystalline sodium cholesteryl sulfonate Form J is further disclosed. “Substantially pure,” as described herein, generally refers to a form herein that is present without any appreciable amounts, other than potentially trace levels of other forms of crystalline sodium cholesteryl sulfonate. Examples of trace levels include not more than about 10%, 5%, 2%, 1.5%, 1%, 0.5%, 0.25%, 0.1%, or less by weight in total relative to the total amount of crystalline sodium cholesteryl sulfonate present.

This disclosure also relates to pharmaceutical compositions containing crystalline sodium cholesteryl sulfonate as disclosed herein. Such pharmaceutical compositions are comprised of one or more pharmaceutically acceptable excipients and crystalline sodium cholesteryl sulfonate as set forth in the present disclosure. Such pharmaceutical compositions may be administered orally or configured to be delivered as any effective conventional dosage unit forms, including, for example, immediate, slow and timed-release oral preparations, parenterally, topically, nasally, ophthalmically, optically, sublingually, rectally, vaginally, and the like.

The present disclosure further includes mixtures of forms of crystalline sodium cholesteryl sulfonate. For examples, mixtures of two or more of sodium cholesteryl sulfonate Form A, crystalline sodium cholesteryl sulfonate Form F, and/or crystalline sodium cholesteryl sulfonate Form J, are provided. The amount of each form present in such mixtures ranges from, for example, about 0.1% to about 99.9% by weight. Other ranges include about 0.1% to about 95%, about 0.1% to about 90%, about 0.1% to about 85%, about 0.1% to about 80%, about 0.1% to about 75%, about 0.1% to about 70%, about 0.1% to about 65%, about 0.1% to about 60%, about 0.1% to about 55%, about 0.1% to about 50%, about 0.1% to about 45%, about 0.1% to about 40%, about 0.1% to about 35%, about 0.1% to about 30%, about 0.1% to about 25%, about 0.1% to about 20%, about 0.1% to about 15%, and about 0.1% to about 10% by weight. Other ranges include about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2%, and about 0.1% to about 1% by weight. Additional ranges include about 0.1% to about 0.9%, about 0.1% to about 0.8%, about 0.1% to about 0.7%, about 0.1% to about 0.6%, about 0.1% to about 0.5%, about 0.1% to about 0.4%, about 0.1% to about 0.3%, and about 0.1% to about 0.2% by weight. Such mixtures may also be present in pharmaceutical compositions for the comprising one or more pharmaceutically acceptable excipients.

The present disclosure further includes methods and uses for treating diseases in humans such as one or more of hypercholesterolemia, hypertriglyceridemia, and conditions related to fat-accumulation and inflammation (e.g., non-alcoholic fatty liver disease (NAFLD), non-alcoholic steatohepatitis (NASH), alcoholic hepatitis, acute kidney injury (AM), acute lung injury (ALI), multi-organ injury, metabolic disorders/disease, diabetes, psoriasis, and atherosclerosis) with effective amounts of crystalline sodium cholesteryl sulfonate and/or pharmaceutical compositions comprising crystalline sodium cholesteryl sulfonate of the present disclosure.

The present disclosure may be further described by one or more of the non-limiting clauses that follow.

Clause 1. Crystalline sodium cholesteryl sulfonate.

Clause 2. A hydrate of crystalline sodium cholesteryl sulfonate.

Clause 3. A solvate of crystalline sodium cholesteryl sulfonate.

Clause 4. The solvate of clause 3, wherein the solvent is an alcohol.

Clause 5. The solvate of clause 4, wherein the alcohol is selected from ethanol and isopropanol.

Clause 6. Crystalline sodium cholesteryl sulfonate Form F.

Clause 7. A solvate of crystalline sodium cholesteryl sulfonate Form F.

Clause 8. The solvate of crystalline sodium cholesteryl sulfonate Form F, wherein the solvent is DMA, MTBE, DMF, or NMP, or a combination thereof.

Clause 9. Crystalline sodium cholesteryl sulfonate of any one of clauses 1, 3, 6, 7, or 8, having an x-ray powder diffraction pattern comprising a peak at about 3.6°2θ.

Clause 10. Crystalline sodium cholesteryl sulfonate of any one of clauses 6 to 9, having an x-ray powder diffraction pattern further comprising one or more peaks at about 7.2°2θ, at about 8.3°2θ, at about 8.9°2θ, at about 9.8°2θ, at about 10.8°2θ, and at about 12.2°2θ.

Clause 11. Crystalline sodium cholesteryl sulfonate of clause 9, having an x-ray powder diffraction pattern comprising a peak at about 7.2°2θ.

Clause 12. Crystalline sodium cholesteryl sulfonate of clause 10 or 11, having an x-ray powder diffraction pattern comprising a peak at about 8.3°2θ.

Clause 13. Crystalline sodium cholesteryl sulfonate of any one of clauses 10 to 12, having an x-ray powder diffraction pattern comprising a peak at about 8.9°2θ.

Clause 14. Crystalline sodium cholesteryl sulfonate of any one of clauses 10 to 13, having an x-ray powder diffraction pattern comprising a peak at about 9.8°2θ.

Clause 15. Crystalline sodium cholesteryl sulfonate of any one of clauses 10 to 14, having an x-ray powder diffraction pattern comprising a peak at about 10.8°2θ.

Clause 16. Crystalline sodium cholesteryl sulfonate of any one of clauses 10 to 15, having an x-ray powder diffraction pattern comprising a peak at about 12.2°2θ.

Clause 17. Crystalline sodium cholesteryl sulfonate of clause 10, having an x-ray powder diffraction pattern comprising a peak at about 3.6°2θ, a peak at about 7.2°2θ, and a peak at about 8.3°2θ.

Clause 18. Crystalline sodium cholesteryl sulfonate of clause 17, further comprising a peak at about 8.9°2θ.

Clause 19. Crystalline sodium cholesteryl sulfonate of clause 17 or clause 18, further comprising a peak at about 10.8°2θ.

Clause 20. Crystalline sodium cholesteryl sulfonate of any one of clauses 1, 3, 6, 7 or 8, having an x-ray powder diffraction pattern comprising a peak at about 10.8°2θ and one or more peaks at about 3.6°2θ, at about 7.2°2θ, at about 8.3°2θ, at about 8.9°2θ, at about 9.8°2θ, and at about 12.2°2θ.

Clause 21. Crystalline sodium cholesteryl sulfonate of clause 20, having an x-ray powder diffraction pattern comprising a peak at about 10.8°2θ and a peak at about 7.2°2θ.

Clause 22. Crystalline sodium cholesteryl sulfonate of clause 20, having an x-ray powder diffraction pattern comprising a peak at about 10.8°2θ and a peak at about 8.3°2θ.

Clause 23. Crystalline sodium cholesteryl sulfonate of clause 20, having an x-ray powder diffraction pattern comprising a peak at about 10.8°2θ and a peak at about 8.9°2θ.

Clause 24. Crystalline sodium cholesteryl sulfonate of clause 20, having an x-ray powder diffraction pattern comprising a peak at about 10.8°2θ and a peak at about 9.8°2θ.

Clause 25. Crystalline sodium cholesteryl sulfonate of clause 20, having an x-ray powder diffraction pattern comprising a peak at about 10.8°2θ and a peak at and about 12.2°2θ.

Clause 26. Crystalline sodium cholesteryl sulfonate of clause 20, having an x-ray powder diffraction pattern comprising a peak at about 10.8°2θ, a peak at about 7.2°2θ, and a peak at about 8.3°2θ.

Clause 27. Crystalline sodium cholesteryl sulfonate of any one of clauses 1, 3, 6, 7, or 8, having an x-ray powder diffraction pattern comprising a peak at about 10.8°2θ, a peak at about 7.2°2θ and a peak at about 8.9°2θ.

Clause 28. Crystalline sodium cholesteryl sulfonate having a DSC thermogram onset temperature of about 165° C.

Clause 29. Crystalline sodium cholesteryl sulfonate of any one of clauses 1, 3, 6, 7, 8, 9, or 10-27, having a DSC thermogram onset temperature of about 165° C.

Clause 30. Crystalline sodium cholesteryl sulfonate of any one of clauses 1-8 or 28, having an x-ray powder diffraction pattern substantially the same as that found in FIG. 2.

Clause 31. An anhydrate of crystalline sodium cholesteryl sulfonate.

Clause 32. Crystalline sodium cholesteryl sulfonate Form J.

Clause 33. Crystalline sodium cholesteryl sulfonate of any one of clauses 1, 31, or 32, having an x-ray powder diffraction pattern comprising a peak at about 2.8°2θ.

Clause 34. Crystalline sodium cholesteryl sulfonate of any one of clauses 1, 31-33, having an x-ray powder diffraction pattern comprising a peak at about 2.8°2θ and one or more peaks at about 2.3°2θ, at about 3.8°2θ, at about 4.7°2θ, at about 14.6°2θ, and at about 16.1°2θ.

Clause 35. Crystalline sodium cholesteryl sulfonate of clause 34, having an x-ray powder diffraction pattern comprising a peak at about 2.3°2θ.

Clause 36. Crystalline sodium cholesteryl sulfonate of clause 34, having an x-ray powder diffraction pattern comprising a peak at about 3.8°2θ.

Clause 37. Crystalline sodium cholesteryl sulfonate of clause 34, having an x-ray powder diffraction pattern comprising a peak at about 4.7°2θ.

Clause 38. Crystalline sodium cholesteryl sulfonate of clause 34, having an x-ray powder diffraction pattern comprising a peak at about 14.6°2θ.

Clause 39. Crystalline sodium cholesteryl sulfonate of clause 34, having an x-ray powder diffraction pattern comprising a peak at about 16.1°2θ.

Clause 40. Crystalline sodium cholesteryl sulfonate of any one of clauses 1, 31, or 32 having an x-ray powder diffraction pattern comprising a peak at about 2.8°2θ and a peak at about 16.1°2θ, further comprising a peak at about 4.7°2θ.

Clause 41. Crystalline sodium cholesteryl sulfonate of any one of clauses 1, 31, or 32 having an x-ray powder diffraction pattern comprising a peak at about 2.8°2θ and a peak at about 16.1°2θ, further comprising a peak at about 7.7°2θ.

Clause 42. Crystalline sodium cholesteryl sulfonate of any one of clauses 1, 31, or 32 having an x-ray powder diffraction pattern comprising a peak at about 2.8°2θ and a peak at about 16.1°2θ, further comprising a peak at about 14.6°2θ.

Clause 43. Crystalline sodium cholesteryl sulfonate of clause 1 or clause 32, having an x-ray powder diffraction pattern substantially the same as that found in FIG. 5.

Clause 44. A mesophase of sodium cholesteryl sulfonate.

Clause 45. Sodium cholesteryl sulfonate Form A.

Clause 46. Sodium cholesteryl sulfonate of clause 44 or clause 45, having an x-ray powder diffraction pattern comprising a peak at about 2.3°2θ.

Clause 47. Sodium cholesteryl sulfonate having a DSC thermogram peak temperature of about 81° C.

Clause 48. Sodium cholesteryl sulfonate of any one of clauses 44, 45, or 46, having a DSC peak temperature of about 81° C.

Clause 49. Sodium cholesteryl sulfonate of any one of clauses 44, 45, or 47, having an x-ray powder diffraction pattern substantially the same as FIG. 1.

Clause 50. A process for making crystalline sodium cholesteryl sulfonate Form J comprising the step of treating sodium cholesteryl sulfonate with isopropanol.

Clause 51. The process of clause 50, further comprising the step of isolating crystalline sodium cholesteryl sulfonate Form J.

Clause 52. The process of clause 51, wherein the crystalline sodium cholesteryl sulfonate Form J is dried.

Clause 53. A mixture of two or more of solid forms of sodium cholesteryl sulfonates.

Clause 54. A mixture of two or more of solid forms of crystalline sodium cholesteryl sulfonates.

Clause 55. A mixture of two or more of sodium cholesteryl sulfonate Form A, crystalline sodium cholesteryl sulfonate Form F, and crystalline sodium cholesteryl sulfonate Form J.

Clause 56. A pharmaceutical composition comprising sodium cholesteryl sulfonate.

Clause 57. A pharmaceutical composition comprising crystalline sodium cholesteryl sulfonate.

Clause 58. The pharmaceutical composition of clause 56 or clause 57 further comprising one or more pharmaceutically acceptable excipients.

Clause 59. A pharmaceutical composition comprising one or more of sodium cholesteryl sulfonate Form A, crystalline sodium cholesteryl sulfonate Form F, and crystalline sodium cholesteryl sulfonate Form J, and one or more pharmaceutically acceptable excipients.

Clause 60. A method of treating or preventing one or more of hypercholesterolemia, hypertriglyceridemia, and conditions related to fat-accumulation and inflammation, for example, non-alcoholic fatty liver disease (NAFLD), non-alcoholic steatohepatitis (NASH), alcoholic hepatitis, acute kidney injury (AM), acute lung injury (ALI), multi-organ injury, metabolic disorders/disease, diabetes, psoriasis, or atherosclerosis, comprising administering to a patient in need thereof an effective amount of a compound or pharmaceutical composition thereof comprising sodium cholesteryl sulfonate.

Clause 61. A method of treating or preventing one or more of hypercholesterolemia, hypertriglyceridemia, and conditions related to fat-accumulation and inflammation, for example, non-alcoholic fatty liver disease (NAFLD), non-alcoholic steatohepatitis (NASH), alcoholic hepatitis, acute kidney injury (AM), acute lung injury (ALI), multi-organ injury, metabolic disorders/disease, diabetes, psoriasis, or atherosclerosis, comprising administering to a patient in need thereof an effective amount of a compound or pharmaceutical composition thereof comprising sodium cholesteryl sulfonate of any one of clauses 1-49, clauses 53-55 and clauses 76-83.

Clause 62. The method of clause 60 or clause 61, wherein the sodium cholesteryl sulfonate is crystalline.

Clause 63. A method of treating or preventing one or more of hypercholesterolemia, hypertriglyceridemia, and conditions related to fat-accumulation and inflammation, for example, non-alcoholic fatty liver disease (NAFLD), non-alcoholic steatohepatitis (NASH), alcoholic hepatitis, acute kidney injury (AM), acute lung injury (ALI), multi-organ injury, metabolic disorders/disease, diabetes, psoriasis, or atherosclerosis, comprising administering to a patient in need thereof an effective amount of a compound or pharmaceutical composition thereof comprising one or more of sodium cholesteryl sulfonate Form A, crystalline sodium cholesteryl sulfonate Form F, and crystalline sodium cholesteryl sulfonate Form J.

Clause 64. A process for preparing crystalline sodium cholesteryl sulfonate Form F comprising the steps of first treating sodium cholesteryl sulfonate in a first suitable solvent to form a mixture; evaporating the suitable solvent from the mixture to form a composition; treating the composition with a second suitable solvent to form a suspension; filtering the suspension to form a material; and drying the material to provide crystalline sodium cholesteryl sulfonate Form F.

Clause 65. The process of clause 64, wherein the first suitable solvent is NMP.

Clause 66. The process of clause 64 or clause 65, wherein the second suitable solvent is MTBE.

Clause 67. Stable crystalline sodium cholesteryl sulfonate.

Clause 68. Stable non-crystalline sodium cholesteryl sulfonate.

Clause 69. Stable sodium cholesteryl sulfonate Form A.

Clause 70. Stable crystalline sodium cholesteryl sulfonate Form F.

Clause 71. Stable crystalline sodium cholesteryl sulfonate Form J.

Clause 72. Use of sodium cholesteryl sulfonate one or more pharmaceutically acceptable excipients, for treating a host mammal with hypercholesterolemia, hypertriglyceridemia, and conditions related to fat-accumulation and inflammation, for example, non-alcoholic fatty liver disease (NAFLD), non-alcoholic steatohepatitis (NASH), alcoholic hepatitis, acute kidney injury (AM), acute lung injury (ALI), multi-organ injury, metabolic disorders/disease, diabetes, psoriasis, or atherosclerosis.

Clause 73. The use of clause 72, wherein sodium cholesteryl sulfonate is crystalline sodium cholesteryl sulfonate.

Clause 74. Use of one or more of sodium cholesteryl sulfonate Form A, crystalline sodium cholesteryl sulfonate Form F, or crystalline sodium cholesteryl sulfonate Form J, and optionally one or more pharmaceutically acceptable excipients, for treating a host mammal with hypercholesterolemia, hypertriglyceridemia, and conditions related to fat-accumulation and inflammation, for example, non-alcoholic fatty liver disease (NAFLD), non-alcoholic steatohepatitis (NASH), alcoholic hepatitis, acute kidney injury (AKI), acute lung injury (ALI), multi-organ injury, metabolic disorders/disease, diabetes, psoriasis, or atherosclerosis.

Clause 75. The use of any one of clauses 72-74, wherein the sodium cholesteryl sulfonate is that of clauses 1-49, clauses 53-55, and clauses 76-83.

Clause 76. Non-crystalline sodium cholesteryl sulfonate.

Clause 77. A hydrate of non-crystalline sodium cholesteryl sulfonate.

Clause 78. A solvate of non-crystalline sodium cholesteryl sulfonate.

Clause 79. Substantially pure crystalline sodium cholesteryl sulfonate.

Clause 80. Substantially pure non-crystalline sodium cholesteryl sulfonate.

Clause 81. Substantially pure sodium cholesteryl sulfonate Form A.

Clause 82. Substantially pure crystalline sodium cholesteryl sulfonate Form F.

Clause 83. Substantially pure crystalline sodium cholesteryl sulfonate Form J.

Clause 84. A process for preparing crystalline sodium cholesteryl sulfonate Form J comprising the steps of treating sodium cholesteryl sulfonate Form A in 1-propanol and drying to make crystalline sodium cholesteryl sulfonate Form J.

Clause 85. A process for preparing sodium cholesteryl sulfonate Form A comprising the steps of Example 1.

Clause 86. Sodium cholesteryl sulfonate as defined in any one of clauses 1 to 49, 67 to 71 and 76 to 83, a mixture of sodium cholesteryl sulfonate as defined in any one of clauses 53 to 55, or a pharmaceutical composition as defined in any one of clauses 56 to 59, for use as a medicament.

Clause 87. Sodium cholesteryl sulfonate as defined in any one of clauses 1 to 49, 67 to 71 and 76 to 83, a mixture as defined in any one of clauses 53 to 55, or a pharmaceutical composition as defined in any one of clauses 56 to 59, for use in a method of treating or preventing one or more of hypercholesterolemia, hypertriglyceridemia, and conditions related to fat-accumulation and inflammation, for example, non-alcoholic fatty liver disease (NAFLD), non-alcoholic steatohepatitis (NASH), alcoholic hepatitis, acute kidney injury (AM), acute lung injury (ALI), multi-organ injury, metabolic disorders/disease, diabetes, psoriasis, or atherosclerosis.

### EXAMPLES

### Example 1—Preparation of Sodium Cholesteryl Sulfonate Form A

Carbon tetrabromide (25.7 g, 77.6 mmol) was added to a mixture of cholesterol (20.0 g, 51.7 mmol) and triphenylphosphine (20.4 g, 77.6 mmol) in dichloromethane (200 mL) at 0° C. The mixture was stirred at room temperature for 10 h. The mixture was diluted with hexanes (100 mL) and filtered through a pad of silica gel. The silica pad was further washed with hexanes. The filtrate was concentrated to provide (3S,8S,9S,10R,13R,14S,17R)-3-bromo-17-[(1R)-1,5-dimethylhexyl]-10,13-dimethyl-2,3,4,7,8,9,11,12,14,15,16,17-dodecahydro-1H-cyclopenta[a]phenanthrene (33.0 g, 70.3% pure, 100%). (3S,8S,9S,10R,13R,14S,17R)-3-Bromo-17-[(1R)-1,5-dimethylhexyl]-10,13-dimethyl-2,3,4,7,8,9,11,12,14,15,16,17-dodecahydro-1H-cyclopenta[a]phenanthrene was diluted with iPrOH (170 mL). Thiourea (19.7 g, 258 mmol) was added to the mixture, and the solution was refluxed for 48 h. Ice (20.0 g) was added to the mixture at 0° C., and the mixture was filtered. The solid was washed with a 2:1 mixture of acetone/water (400 mL), acetone (10.0 mL), and hexanes (200 mL) and dried under reduced pressure to form [amino-[[(3S,8S,9S, 10R,13R,14S,17R)-17-[(1R)-1,5-dimethylhexyl]-10,13-dimethyl-2,3,4,7,8,9,11,12,14,15,16,17-dodecahydro-1H-cyclopenta[a]phenanthren-3-yl]sulfanyl]methylene]ammonium;bromide as solid (25.7 g, 95%). 1H NMR (500 MHz, DMSO) δ 9.01 (d, J=79.8 Hz, 4H), 5.40 (s, 1H), 3.63 (t, J=13.0 Hz, 1H), 2.34 (td, J=12.6, 7.3 Hz, 2H), 2.03-1.71 (m, 5H), 1.71-0.75 (m, 33H), 0.66 (s, 3H).

NaOH (5.86 g, 147 mmol) was added to a solution of [amino-[[(3 S,8 S,9S,10R,13R,14S,17R)-17-[(1R)-1,5-dimethylhexyl]-10,13-dimethyl-2,3,4,7,8,9,11,12,14,15,16,17-dodecahydro-1H-cyclopenta[a] phenanthren-3-yl]sulfanyl]methylene]ammonium;bromide (25.7 g, 48.9 mmol) in EtOH (400 mL) at 0° C. The mixture was stirred at 22° C. for 4 h and was neutralized (pH 7) with HCl (1 M in water). The aqueous phase was extracted with hexanes (3×300 mL), and the combined organic phases were washed with water (300 mL), dried (Na2SO4), filtered, and concentrated. The residue was filtered through silica gel, eluting with hexanes and EtOAc (9:1), and the filtrate was concentrated to provide (3S,8S,9S,10R,13R,14S,17R)-17-[(1R)-1,5-dimethylhexyl]-10,13-dimethyl-2,3,4,7,8,9,11,12,14,15,16,17-dodecahydro-1H-cyclopenta[a]phenanthrene-3-thiol as a solid (14.1 g, 71%). 1H NMR (500 MHz, CDCl3) δ 5.25 (d, J=4.6 Hz, 1H), 2.69-2.54 (m, 1H), 2.24 (d, J=7.6 Hz, 2H), 2.01-1.70 (m, 5H), 1.60-0.74 (m, 34H), 0.60 (s, 3H).

Peracetic acid (1.67 mL, 7.9 mmol, 32% in AcOH) was slowly added to a solution of (3S,8S,9S,10R,13R,14S,17R)-17-[(1R)-1,5-dimethylhexyl]-10,13-dimethyl-2,3,4,7,8,9,11,12,14,15,16,17-dodecahydro-1H-cyclopenta[a]phenanthrene-3-thiol (1.00 g, 2.50 mmol) and 2-methyl-2-butene (658 μL, 6.20 mmol) in CH₂Cl₂ (12.5 mL) at 0° C. The mixture was stirred at 0° C. for 2 h and filtered. The solid was washed with CH₂Cl₂ (20.0 mL) and diluted with sat. aq. NaHCO₃ (20.0 mL). The mixture was filtered, and the solid was washed with a mixture of water and brine (1:1, 15.0 mL), water (10.0 mL), and CH₂Cl₂ (10.0 mL). The solid was dried under vacuum to provide the title compound as a solid (579 mg, 49%). ¹H NMR (400 MHz, DMSO-d₆) δ 5.23 (d, J=3.7 Hz, 1H), 2.20 (d, J=7.4 Hz, 2H), 2.10-2.05 (m, 1H), 1.98-1.68 (m, 5H), 1.60-0.71 (m, 33H), 0.62 (s, 3H).

In this Example 1, all temperatures are in degrees Celsius (° C.) and are uncorrected. Reagent grade chemicals and anhydrous solvent were purchased from commercial sources and unless otherwise mentioned, were used without further purification. Silica gel chromatography was performed on Teledyne Isco instruments using pre-packaged disposable SiO₂ stationary phase columns with eluent flow rate range of 15 to 200 mL/min, UV detection (254 and 280 nm). Reverse phase preparative HPLC was carried out using C18 columns, UV detection (214 and 254 nm) eluting with gradients of MeCN in H₂O (0.03% (NH₄)₂CO_{3/}0.375% NH₄OH) (high pH), MeCN in H₂O (0.1% HCOOH) (low pH) or MeCN in H₂O (neutral pH). The analytical HPLC chromatograms were performed using an Agilent 1100 series instrument with DAD detector (190 nm to 300 nm). The mass spectra were recorded with a Waters Micromass ZQ detector at 130° C. The mass spectrometer was equipped with an electrospray ion source (ESI) operated in a positive ion mode and was set to scan between m/z 150-750 with a scan time of 0.3 s. Products and intermediates were analyzed by HPLC/MS on a Gemini-NX (5 uM, 2.0×30 mm) using a high pH buffer gradient of 5% to 100% of MeCN in H₂O (0.03% (NH₄)₂CO₃/0.375% NH₄OH) over 2.5 min at 1.8 mL/min for a 3.5 min run (B05) and EVO C18 (5 uM, 3.0×50 mm) using a low pH buffer gradient of 5% to 100% of MeCN in H₂O (0.1% HCOOH) over 2.5 min at 2.2 mL/min for a 3.5 min run (A05). The ¹H NMR spectra were recorded on a Bruker UltraShield 500 MHz/54 mm instrument (BZH 43/500/70B, D221/54-3209). The chemical shifts are referenced to solvent peaks, which in ¹H NMR appears at 7.26 ppm for CDCl₃, 2.50 for DMSO-d6, and 3.31 ppm for CD₃OD.

### Example 2—Preparation of Crystalline Sodium Cholesteryl Sulfonate Form F

A mixture of 76.2 mg of sodium cholesteryl sulfonate of Example 1 in 0.5 mL of N-methyl-2-pyrrolidone (NMP) was magnetically stirred at ambient conditions for 5 days. The solvent was then allowed to evaporate freely from the mixture in an open vessel at ambient conditions overnight. A white suspension was then generated with the addition of 1 mL of methyl tert-butyl ether (MTBE), which was then magnetically stirred at about 53° C. for 12 days. Sticky and damp material was recovered from the suspension with water-aspirated vacuum filtration. A portion of the material was dried at about 40° C. under vacuum for 3 days, providing approximately 34.7 mg of crystalline sodium cholesteryl sulfonate Form F.

### Example 3—Preparation of Crystalline Sodium Cholesteryl Sulfonate Form J

A gel-like viscous liquid generated with 50.7 mg of sodium cholesteryl sulfonate Form A of Example 1 in 1 mL of 1-propanol (1-PrOH) was magnetically stirred at approximately 65° C. for 2 days. The viscous liquid was then allowed to evaporate to dryness overnight at 65° C. FIG. 5 is an x-ray powder diffraction pattern of crystalline sodium cholesteryl sulfonate Form J.

### Example 4—Instrumental Techniques

### X-Ray Powder Diffraction (XRPD)

XRPD patterns were collected with a PANalytical X'Pert PRO MPD diffractometer or a PANalytical Empyrean diffractometer using an incident beam of Cu radiation produced using an Optix long, fine-focus source. An elliptically graded multilayer mirror was used to focus Cu Kα X-ray radiation through the specimen and onto the detector. Prior to the analysis, a silicon specimen (NIST SRM 640e) was analyzed to verify the observed position of the Si (111) peak is consistent with the NIST-certified position. A specimen of the sample was sandwiched between 3-μm-thick films and analyzed in transmission geometry. A beam-stop, short antiscatter extension, and antiscatter knife edge were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v. 2.2b or software v. 5.5.

### Differential Scanning Alorimetry (DSC)

DSC was performed using a Mettler-Toledo DSC3+ differential scanning calorimeter. A tau lag adjustment is performed with indium, tin, and zinc. The temperature and enthalpy were adjusted with octane, phenyl salicylate, indium, tin, and zinc. The adjustment was then verified with octane, phenyl salicylate, indium, tin, and zinc. The sample was placed into a hermetically sealed aluminum DSC pan, and the weight was accurately recorded. The pan lid was pierced by the instrument and then inserted into the DSC cell for analysis. A weighed aluminum pan configured as the sample pan was placed on the reference side of the cell. The temperature heating ramp rate was at ten kelvin per minute.

### Thermogravimetry (TGA) and TGA/DSC Combination Analysis

TGA and TGA/DSC combination analyses were performed using a Mettler-Toledo TGA/DSC3+ analyzer. Temperature and enthalpy adjustments were performed using indium, tin, and zinc, and then verified with indium. The balance was verified with calcium oxalate. The sample was placed in an open aluminum pan. The pan was hermetically sealed, the lid pierced, then inserted into the TG furnace. A weighed aluminum pan configured as the sample pan was placed on the reference platform. The furnace was heated under nitrogen.

### Hot Stage Microscopy (HSM)

Hot stage microscopy was performed using a Linkman hot stage (model FTIR 600) with a TMS93 controller on a Leica DM LP microscope. Sample was observed using 10×0.22 N.A. long working distance objective with a lambda plate with crossed polarizers. Sample was placed on a coverslip. Another coverslip was then placed over the sample. Sample was visually observed as the stage was heated. Images were captured using a SPOT Insight™ color digital camera with SPOT Software v. 4.5.9. The hot stage was calibrated using USP melting point standards.

### Solution Proton Nuclear Magnetic Resonance Spectroscopy (¹H NMR)

Solution proton NMR spectra were acquired by an Agilent DD2-400 spectrometer or with a Varian ^{UNITY}INOVA-400 spectrometer.

### XRPD Indexing

The high-resolution XRPD patterns were indexed using X'Pert High Score Plus 2.2a (2.2.1) in this study. Indexing and structure refinement are computational studies. Agreement between the allowed peak positions, marked with red bars, and the observed peaks indicates a consistent unit cell determination. Successful indexing of the pattern indicates that the sample is composed primarily of a single crystalline phase. Space groups consistent with the assigned extinction symbol, unit cell parameters, and derived quantities are tabulated below each figure showing tentative indexing solution. To confirm a tentative indexing solution, the molecular packing motifs within the crystallographic unit cells should be determined, however no attempts at molecular packing were performed herein.

1. Crystalline sodium cholesteryl sulfonate.

2. A hydrate of crystalline sodium cholesteryl sulfonate.

3. A solvate of crystalline sodium cholesteryl sulfonate.

4. Crystalline sodium cholesteryl sulfonate Form F.

5. A solvate of crystalline sodium cholesteryl sulfonate Form F.

6. A solvate of crystalline sodium cholesteryl sulfonate Form F, wherein the solvent is DMA, MTBE, DMF, or NMP, or a combination thereof.

7. Crystalline sodium cholesteryl sulfonate having a DSC thermogram onset temperature of about 165° C.

8. An anhydrate of crystalline sodium cholesteryl sulfonate.

9. Crystalline sodium cholesteryl sulfonate Form J.

10. A mesophase of sodium cholesteryl sulfonate.

11. Sodium cholesteryl sulfonate Form A.

12. Sodium cholesteryl sulfonate having a DSC thermogram peak temperature of about 81° C.

13. A pharmaceutical composition comprising sodium cholesteryl sulfonate.

14. A pharmaceutical composition comprising crystalline sodium cholesteryl sulfonate.

15. A pharmaceutical composition comprising one or more of sodium cholesteryl sulfonate Form A, crystalline sodium cholesteryl sulfonate Form F, and crystalline sodium cholesteryl sulfonate Form J, and one or more pharmaceutically acceptable excipients.

16. Non-crystalline sodium cholesteryl sulfonate.

17. A hydrate of non-crystalline sodium cholesteryl sulfonate.

18. A solvate of non-crystalline sodium cholesteryl sulfonate.
